(19) United States
(12) Patent Application Publication
(10) Pub. No.: US 2002/0015668 A1
(43) Pub. Date: Feb. 7, 2002

(54) Device for the atomization of cleaning and disinfecting liquids

(76) Inventors: Lucio Dell'Acqua, Viadana (IT); Michelotti Luca, Montecchio Emilia (IT)

Correspondence Address: James V. Costigan, Esq., HEDMAN & COSTIGAN, P.C., Suite 2003, 1185 Avenue of the Americas, New York, NY, 10036-2646, US

(21) Appl. No.: 09/911,015

(22) Filed: Jul. 23, 2001

(30) Foreign Application Priority Data
Jul. 31, 2000 (IT) MI2000A 001768

(51) Int. Cl. A61L 9/00

(52) U.S. Cl. 422/124; 422/123; 422/120; 422/305

(57) ABSTRACT

A device (10) for the atomization of cleaning and disinfecting liquids, comprises a portable container (11), divided into at least a first and second compartment (14, 15), wherein inside the first compartment (14) there is a body (16), which has an inlet channel (17) for a cleaning and/or disinfecting liquid, and means for grasping the neck of the cleaning and/or disinfecting liquid jar (20). On the bottom of the body (16) for containing and dosing the above liquid, there are piezoelectric elements (24), activated by an electronic circuit (25) in order to create the immediate atomization of the liquid to be applied. The device is also equipped with an exit channel (18), for the atomized liquid and the vapor generated starting from said liquid.

The present invention relates to a device for the atomization of cleaning and disinfecting liquids.

There are three types of common devices used for the application of disinfecting liquid products.

In the first place, the application is effected by maintaining the product in the liquid state and creating a flow thereof, with deposition due to the friction between the liquid and the surface onto which it flows.

To implement systems of this kind, however, it is always necessary to create a flow of liquid by using, for example, a hydraulic pump or compressor.

In a second type of known device, the application of cleaning and disinfecting liquid is effected by bringing the product to a high temperature to obtain hot vapour, which is then applied by means of various methods.

In systems of this kind, however, condensation of the liquid may take place, normally due to a high temperature differential, which causes the liquid to be deposited too easily onto the surfaces which it encounters on the way.

It is also necessary to be equipped with heating devices.

Alternatively, in a third type of known device, the product is contained in appropriate cylinders from which the liquid is extracted with spray atomizers (for example aerosol sprays).

In systems of this type, however, the particles may be too heavy, causing them to be too rapidly deposited.

Furthermore, a high pressure must be created for the functioning of spray-type atomizers.

The objective of the present invention is consequently to overcome these problems, by means of a device for the atomization of cleaning and disinfecting liquids which allows an extremely uniform deposition of the product, in both a simple and economic way.

These and other objectives are reached by means of a device for the atomization of cleaning and disinfecting liquids, according to claim 1, to which reference should be made for the sake of brevity.

Further characteristics of the invention are defined in the other claims enclosed with the present application.

Additional objectives and advantages of the present invention will appear evident from the following description and enclosed drawings, which are provided for purely illustrative purposes but which do not limit the scope of the invention, wherein:

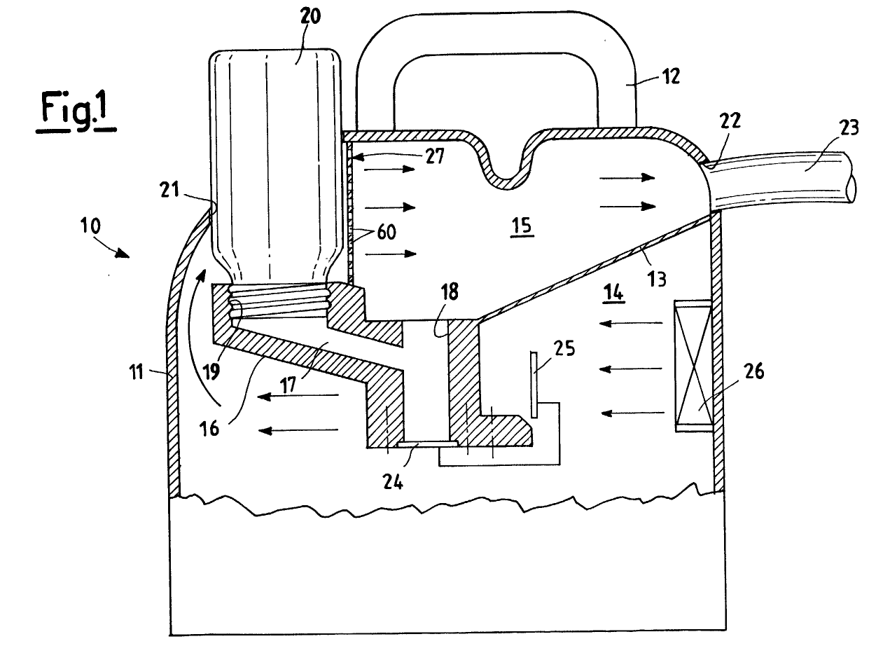

FIG. 1 represents a partially sectional view of a device for the atomization of cleaning and disinfecting liquids, according to the present invention;

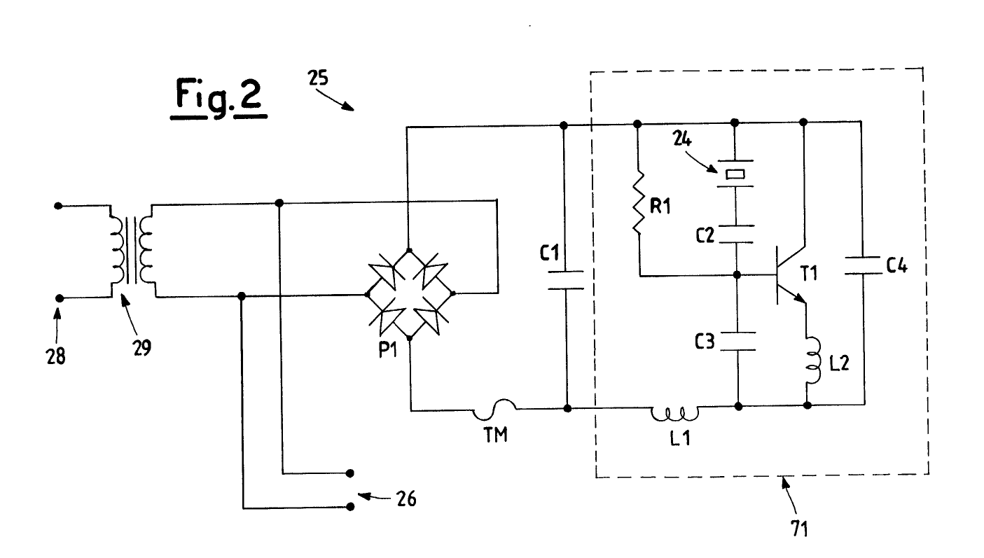

FIG. 2 represents a scheme relating to the electronic circuit which allows the atomization of the cleaning and disinfecting liquids, according to the invention; and

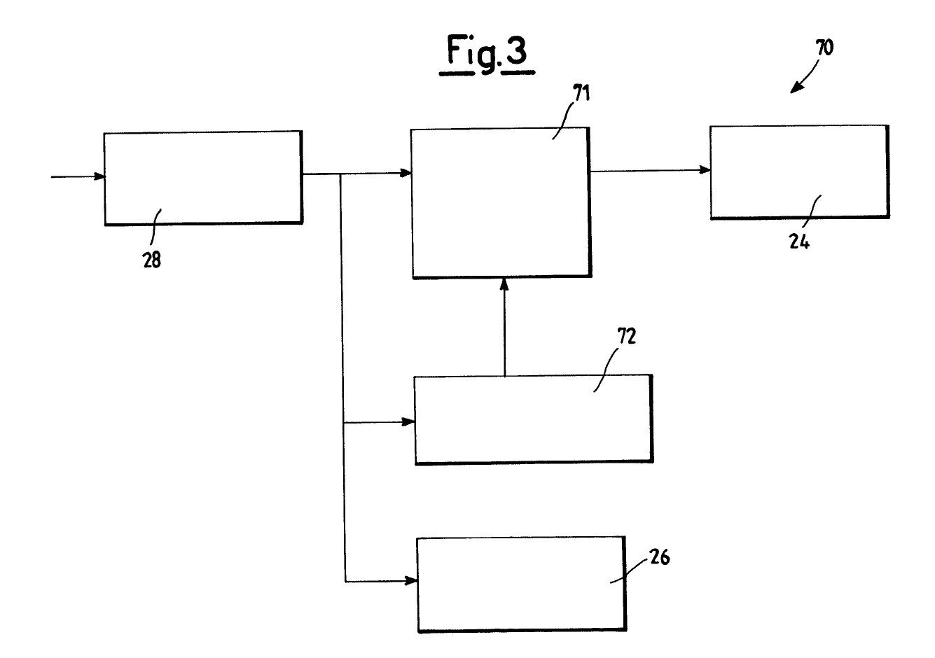

FIG. 3 represents a block scheme relating to the device for the atomization of liquids according to the invention.

With particular reference to the above figures, the device for the atomization of cleaning and disinfecting liquids, according to the present invention, is indicated as a whole by the reference number 10.

The device 10 is made up of a case-like container 11, preferably equipped with a handle 12, and divided by a partition 13, into two compartments 14 and 15.

Inside the compartment 14, there is a body 16, which can be made of plastic or metallic materials and which has an inlet channel 17 for a cleaning and/or disinfecting liquid, and an exit channel 18, for the atomized liquid and vapour generated starting from said liquid.

The body 16 also has a threaded mouth 19 for grasping the neck of a jar 20 containing the cleaning and/or disinfecting liquid.

The case-like container 11, in fact, has a hole 21 for inserting the jar 20 and also an exit hole 22, situated in one of the outer surfaces which forms part of compartment 15, for a flexible tube 23.

The exit hole 22 for the flexible tube 23 is situated in counter-position to the jar 20.

On the bottom of the body 16, which is used for containing and dosing the cleaning and/or disinfecting liquid, some piezoelectric elements 24 are present, for example ceramic vibration or transducer devices which create the immediate atomization of the liquid to be applied, which, in turn, is kept at a constant level above the ceramic transducer.

The electronic circuit 25, illustrated in more detail in FIG. 2, which converts the electric oscillation of the ceramic device 24 to mechanical oscillation at ultrasonic frequency, is also present inside compartment 14.

Inside compartment 14, there is also an electro-fan 26; and also an electric power supply 28, preferably external.

Finally, it can be observed that compartment 14 and compartment 15 are also divided by a perforated wall 27, of which the holes 60 are visible.

FIG. 2 represents the electric scheme, indicated as a whole by reference number 25, relating to the electronic circuit which allows the atomization of cleaning and disinfecting liquids.

The circuit 25 has a pair of clips for removing the voltage from the transformer 19, fed by means of line voltage (for example alternating 220V), from the power supply 28; the electric circuit of the fan 26 is also fed by means of the transformer 29.

The circuit 25 is also equipped with a thermo-resistance TM, which starts functioning if a pre-fixed temperature value is exceeded.

The alternating current is subsequently rectified by means of a diode bridge P1 and stabilized by the condenser C1.

The voltage obtained feeds the subsequent electric oscillator 71 (of which the condensers C2, C3 and C4, the resistance R1, the inductors L1 and L2 and the transistor T1, are visible), which, in its preferred version, has an oscillation frequency of 1.7 Mhz.

This oscillation frequency causes the vibration of the ceramic transducer 24.

FIG. 3 also illustrates a block scheme, indicated as a whole by reference number 70, relating to the device of the invention.

The scheme shows the external power supply 28 which feeds both the electric oscillator 71, the electro-fan 26, and also an electronic floating device 72, in turn capable of communicating to the electronic oscillator 71 a signal which indicates the level of the liquid inside the body 16.

FIG. 3 obviously also demonstrates the fact that the electric oscillator 71 acts on the ceramic transducer 24.

The functioning of the device for the atomization of cleaning and disinfecting liquids, according to the present invention, is briefly illustrated below in the continuation of the present description.

First of all, the cleaning and/or disinfecting liquid jar 20 is inserted inside the case-like container 11, so that it is screwed into the threaded mouth 19 of the metal body 16.

The device 10 is then applied to the place to be cleaned or disinfected, by means of the flexible tube 23, and is activated so that the circuit 25, by means of the ceramic device 24, vaporizes the liquid leaving the jar 20 and which, passing through the duct 17, comes into contact with the ceramic transducers 24.

The liquid which evaporates through the duct 18 is sent into compartment 15, and the fan 26 creates a stream of air which follows the path indicated by the arrows in FIG. 1, first through compartment 14, then turning behind and around the container 20, then through the perforated wall 27, it pushes the vapour through the flexible tube 23 onto the parts to be treated.

At the same time, the fan 26 also enables the cooling of the circuit 25.

In short, the device of the invention, which is considerably different from the types of known techniques described above, finds its main application in the project of a cleaning system for air-conditioning plants, but it can also be used for the application of liquid products in different kinds of plants.

The system uses one or more electronic circuits for converting electric oscillation into mechanical oscillation at ultrasonic frequency, by means of a ceramic vibration device.

The immediate atomization is created of the liquid to be applied, which is kept at a constant level above the ceramic transducer.

An electric detection system of the liquid level interrupts the functioning as soon as it drops below a preestablished level.

The vapourized liquid is immediately sent, by means of a fan, into the flexible application tube which can be positioned and introduced in the plant to be disinfected.

The system in question thus allows the atomization of cleaning and disinfecting liquids and their application on the plant to be treated.

The description provided clearly illustrates the characteristics of the device for the atomization of cleaning and disinfecting liquids, object of the present invention, as also the advantages.

We would like to make the following conclusive comments and observations to more accurately explain and define these advantages.

With the use of the device of the invention, the vapourized liquid is not easily deposited onto the surfaces which it encounters, thus avoiding condensation phenomena normally due to a high temperature differential.

Furthermore, the device of the invention is ideal for use with highly flammable liquids (such as alcohol-based detergents), as there are no high-temperature parts or electric components which can generate sparks along the exit path of the product.

This also allows the use of liquids whose chemical properties would be altered by thermal stress.

The particles atomized with the above system are extremely homogeneous, with a dimension of more or less 5 microns.

This homogeneity allows the product to be extremely uniformly deposited.

The particles, moreover, are sufficiently light as to penetrate the plant in depth before being deposited, unlike what occurs in the use of classical aerosol sprays in which the weight of the atomized particles causes their rapid deposition.

The electric consumption of the system obtained is considerably reduced with respect to the other types of application mentioned in the three previous points, for the following respective reasons.

It is not necessary to create a liquid flow and therefore there is no hydraulic pump or compressor.

It is not necessary to have a heater as the atomization is not obtained by a rise in the temperature of the liquid.

It is not necessary to create a high pressure as is compulsory for the functioning of spray-type atomizers, but it is sufficient to have a small electro-fan for moving extremely light particles.

Finally, it is evident that numerous variations can be applied to the device for the atomization of cleaning and disinfecting liquids, object of the present invention, without excluding any of the novelty principles which characterize the inventive idea.

In the embodiment of the invention, the materials, forms and dimensions of the details illustrated can vary according to the demands and can be substituted with other technically equivalent alternatives.

The scope of the invention is defined in the claims enclosed with the present patent application.

1. A device (10) for the atomization of cleaning and disinfecting liquids, characterized in that it comprises a portable container (11), divided into at least a first and second compartment (14, 15), wherein, inside the first compartment (14) there is a body (16), which has an inlet channel (17) for a cleaning and/or disinfecting liquid, and an exit channel (18), for the atomized liquid and vapour generated starting from said liquid, means for grasping the neck of a cleaning and/or disinfecting liquid jar (20) and wherein, on the bottom of the above body (16) whose purpose is to contain and dose the cleaning and/or disinfecting liquid, there are piezoelectric elements (24), activated by an electronic circuit (25) in order to create the immediate atomization of the liquid to be applied.

2. The device (10), according to claim 1, characterized in that, inside the above first compartment (14) there is the above electronic circuit (25) suitable for converting the electric oscillation of the piezoelectric elements (24) into mechanical oscillation at ultrasonic frequency.

3. The device (10), according to claim 1, characterized in that inside the above first compartment (14) there is an electro-fan (26).

4. The device (10), according to claims 2 or 3, characterized in that the above electronic circuit (25) and the above electro-fan (26) are fed by the same electric power supply (28).

5. The device (10) according to claim 1, characterized in that the above first and second compartments (14, 15) are also divided by a perforated wall (27), through which the stream of air created by the above electro-fan (26) can pass to facilitate the exit of the vapourized liquid.

6. The device (10) according to the previous claims, characterized in that the above body (16) is made of plastic or metallic materials.

7. The device (10) according to the previous claims, characterized in that the above portable container (11) has a handle (12).

8. The device (10) according to the previous claims, characterized in that it has an electronic floating device (72), capable of communicating to the above electronic circuit (25), a signal which indicates the level of the liquid inside the body (16).
